(19) United States
(12) Patent Application Publication
(10) Pub. No.: US 2023/0348556 A1
(43) Pub. Date: Nov. 2, 2023

(54) ARTIFICIAL IMMUNOSURVEILLANCE CHIMERIC ANTIGEN RECEPTOR AND CELLS EXPRESSING THE SAME

(71) Applicants: ARBELE Limited, Shatin, N.T. (HK); Avalon GloboCare Corporation, Freehold, NJ (US)

(72) Inventors: Donald E. Staunton, Kirkland, WA (US); John M. Luk, BELLEVUE, WA (US); John M. Harlan, Seattle, WA (US); David Kin Jin, Staten Island, NY (US)

(21) Appl. No.: 17/432,920

(22) PCT Filed: Feb. 21, 2020
(86) PCT No.: PCT/US2020/019374

§ 371 Date: Aug. 21, 2021

Related U.S. Application Data
(60) Provisional application No. 62/808,833, filed on Feb. 21, 2019.
(60) Provisional application No. 62/808,830, filed on Feb. 21, 2019.
(60) Provisional application No. 62/808,823, filed on Feb. 21, 2019.
(60) Provisional application No. 62/808,815, filed on Feb. 21, 2019.

(51) Int. Cl. C07K 14/725; C07K 14/715; C07K 16/28

(52) U.S. Cl. CPC C07K 14/7051; C07K 14/7155; C07K 16/2887; C07K 2317/31; C07K 2317/622; C12N 15/63

(57) ABSTRACT

A non-viral vector, comprising, from 5′ to 3′, flanked by two transposon terminal inverted repeats, an inducible promoter, a first coding region comprising a specific combination of genes whose expression is localized to a tumor microenviorment and their function being mediators of CAR persistence and anti-tumor mechanisms to stimulate or enhance a patient anti-tumor response; a second promoter and coding region for expressing one or more artificial immunosurveillance chimeric antigen receptors (AI-CAR), and a truncated CD20 or truncated EGFR safety target. A polycistronic mRNA comprised of a specific combination of transiently expressed anti-tumor mechanisms designed to stimulate a patient anti-tumor response the same as an AI-CAR vector.

### CROSS REFERENCE TO RELATED APPLICATIONS

This application claims the benefit of the filing date of U.S. Provisional Application Ser. No. 62/808,815 filed Feb. 21, 2019, U.S. Provisional Application Ser. No. 62/808,823 filed Feb. 21, 2019, U.S. Provisional Application Ser. No. 62/808,830 filed Feb. 21, 2019, and U.S. Provisional Application Ser. No. 62/808,833, filed Feb. 21, 2019 under 35 U.S.C. 119(e), the entire disclosures of which are incorporated by reference herein.

### TECHNICAL FIELD

This invention relates to the technology for improving the expansion, manufacturing, survival and efficacy of chimeric antigen receptor (CAR)-T cells or NK cells.

### BACKGROUND

Chimeric antigen receptor (CAR) is the center piece of immune therapy that brings antibody directed targeting into cellular immunity for treating cancer. A CAR is created to have a tumor antigen binding domain linked to the intracellular domains of TCR and TCR co-stimulatory proteins. After engineering a patient’s T or NK cells to produce the CAR and infusing them back to the patient, the CAR-expressing T cells are equipped to kill cancer cells that express the tumor antigen. The first two FDA approved CAR-T cell therapies are both targeting CD19, which is a pan B-cell surface molecule on many types of B-cell cancers. Kymriah (tisagenlecleucel) is approved for treating relapsed/refractory B-cell precursor acute lymphoblastic leukemia (ALL), while Yescarta (axicabtagene ciloleucel) is approved for treating relapsed/refractory diffuse large B-cell lymphoma (DLBCL). In clinical trials, the result of post Kymriah treatment revealed an 83% remission rate for all types of B-cell ALL after three months. However, 49% of ALL patients suffered cytokine release syndrome (CRS), a serious side effect that has been responsible for multiple deaths in clinical trials. In addition, at least 10% of patients relapsed due to loss of the targeted CD19 epitope. In a CLL trial Kymriah treatment resulted in a 57% overall response rate (Boyiadzis 2018).

Multiple factors may contribute to the relapse of CAR-T therapy, such as insufficient CAR T-cell persistence (due to exhaustion or a host anti-CAR response), loss of target antigen, lack of induction of an effective host anti-tumor response, and inability to efficiently locate to lymphoma/solid tumors. Loss of CD19 expression occurs in B-cell neoplasms and after immunotherapy targeting CD19 (Masir 2006, Kimura 2007, Yu 2017). Loss of tumor antigen may be addressed by targeting a second pan-B cell/tumor antigen, such as CD20 and CD22. In case of CD22, it is broadly expressed on ALL blasts and has been targeted successfully with immunoconjugates and mono-specific CAR T cells (Boyiadzis 2018). One strategy to significantly decrease the chance of immune escape is to target CD22 as a second antigen using a CAR that is bispecific.

Several clinical trials of anti-CD19 x anti-CD22 bispecific CAR are underway (Boyiadzis 2018). The configuration of these CARs is based on FDA-approved second-generation CAR, which have in common a single chain with a CD3ζ endodomain. Although the terms of dual and bispecific are used, all of them are bispecific CARs. If the side effect of CRS results from the infusion of CARs, the addition of anti-CD22 scFv will not make much difference. Nonetheless, it remains unclear how many patients in these trials will experience CRS.

There is a need for a non-viral bispecific CAR expression construct that constitutes a combination therapy that results in a more complete and durable response. In this manner cytokines are provided not only to enhance CAR cytotoxicity, survival and proliferation but also to induce a host anti-tumor response. Moreover, the cytokine response is localized and transient and therefore circumvents toxicities that have been observed with systemic administration of cytokines. In addition to the concerns of systemic cytokine therapy, and loss of tumor antigens, the issues of CAR cell survival, persistence, exhaustion and manufacturing are related. To mount a complete and durable response, there are approaches of supplementing soluble cytokine growth factors, multiple dosing, or activating cytokine signaling pathways in order to drive expansion without terminal differentiation. While cytokine treatment may enhance in vivo expansion and activity of CAR, the production of CAR cells may utilize multiple cytokines for expansion. These issues may be addressed by production of a selected combination of cytokines in response to target antigens.

### SUMMARY

In one aspect, the application provides bispecific chimeric antigen receptors. In one embodiment, such receptor comprises an extracellular domain linked to an intercellular domain through a linker, wherein the extracellular domain comprises a first scFv linked to a second scFv, wherein the first scFv domain and the second scFv domain each independently has affinity towards CD19 or CD22, wherein the first and the second scFv domain has affinity towards different antigens, and wherein the intercellular domain comprises a co-stimulatory endo-domain domain an a CD3ξ domain.

In a second aspect, the application provides dual specific chimeric antigen receptor complexes. In one embodiment, such complex comprises a first protein, comprising a first extracellular domain linked to a first intercellular domain through a first linker, wherein the first extracellular domain comprises a first scFv having affinity towards CD19 or CD22, and wherein the first intercellular domain comprises a JAK1 binding domain, and a second protein, comprising a second extracellular domain linked to a second intercellular domain through a second linker, wherein the second extracellular domain comprises a second scFv having affinity towards CD 19 or CD22, and wherein the second intercellular domain comprises a JAK3 binding domain. The first scFv domain and the second scFv domain has affinity toward different tumor antigens.

In one embodiment, the first intracellular domain comprises IL7Rα(CD127). In one embodiment, the first intracellular domain comprises intracellular domain of IL15Rβ(CD122), IL21Rα (CD360), or a combination thereof. In one embodiment, the first intracellular further comprises a first cytotoxic signaling domain linked to a JAK1 binding domain. In one embodiment, the first cytotoxic signaling domain comprises CD28, CD3ζ, CD137, OX40, CD27, ICOS, or a combination thereof.

In one embodiment, the first scFv domain has an affinity toward CD19.

In one embodiment, the second scFv domain has an affinity toward CD22.

In one embodiment, the first intracellular domain is configured to dimerize with the second intracellular domain.

In one embodiment, the second intracellular domain comprises y(CD132). In one embodiment, the second intracellular domain further comprises a second cytotoxic signaling domain linked to a JAK3 binding domain. In one embodiment, the second intracellular domain comprises in tandem y(CD132), JAK3 binding domain, CD28, and CD3ζ.

In one embodiment, the second cytotoxic domain comprises CD28, CD3ζ, CD137, OX40, CD27, ICOS, or a combination thereof.

In one embodiment, the first and the second linker comprises independently CD8. In one embodiment, the first and the second linker comprises independently a stalk and a transmembrane domain.

In one embodiment, the stalk comprises CD8, Fc hinge, Fc CH2-CH3, TCRα, TCRβ, truncated IL7Rα (CD127), truncated IL15Rβ (CD122), IL15Rα (CD215), truncatedy (CD132), truncated IL21Rα (CD360), or a combination thereof.

In one embodiment, the transmembrane domain comprises CD8, CD28, CD3ζ, CD3ε, CD3δ, CD3γ′, CD3ζ, TCRα, TCRβ, IL15Rβ (CD122), y(CD132), IL7Rα (CD127), IL21Rα (CD360), IL15Rα (CD215), or a combination of.

In another aspect, the application provides open reading frames (ORFs). In one embodiment, the open reading frame (ORF) comprises sequentially a nucleic acid encoding the protein as disclosed thereof, a nucleic acid encoding a ribosomal skipping sequence, and a nucleic acid encoding the protein as disclosed thereof. In one embodiment, the open reading frame (ORF) comprises sequentially CD22 scFv, a linker, CD22 scFv, and a chimeric antigen receptor domain.

In a further aspect, the application provides biomolecule complexes. In one embodiment, such complex comprises the bispecific or dual chimeric antigen receptor as disclosed thereof bound to a CD19 antigen or a CD22 antigen. In one embodiment, the first intracellular domain is dimerized with the second intracellular domain. In one embodiment, JAK1 is dimerized with JAK3.

In a further aspect, the application provides non-viral vectors. In one embodiment, the non-viral vector comprises, from 5′ to 3′, flanked by two transposons, a promoter, a first coding region comprising a gene for expressing a first artificial immunosurveillance chimeric antigen receptor (Al-CAR), a fourth coding region comprising a gene expressing a truncated CD20 or truncated EGFR safety target, followed by a polyA signal sequence.

In one embodiment, the first promoter comprises a STAT, NFAT, or NF-κB inducible promoter. In one embodiment, the first coding region and the fourth coding region is linked by an IRES. In one embodiment, the first AI-CAR comprises CD19 CAR or CD22 CAR.

In one embodiment, the non-viral vector further comprises a second coding region comprising a gene for expressing a second AI-CAR, intermediating the first coding region and the fourth coding region. In one embodiment, the first CAR comprises CD19 CAR and wherein the second region comprises CD22 CAR or CD20 CAR.

In one embodiment, the non-viral vector further comprises a third coding region comprising a gene for expressing a third AI-CAR, intermediating the second coding region and the fourth coding region. In one embodiment, the first CAR comprises CD19scFv, wherein the second CAR comprising CD22 scFv or CD20 scFv, and wherein the first CAR is linked to the second CAR through a linker.

In a further aspect, the application provides the isolated nucleic acids. In one embodiment, the isolated nucleic acid encodes the proteins, receptors, biomolecules, or biomolecule complexes thereof.

In a further aspect, the application provides expression vectors. In one embodiment, the expression vector comprises the isolated nucleic acid as disclosed thereof. In one embodiment, the expression vector comprises a nucleic acid encoding cytokine expression and the ORF as disclosed thereof. In one embodiment, the vector is expressible in a cell.

In a further aspect, the application provides host cell. In one embodiment, the host cell comprises the isolated nucleic acids or the expression vectors as disclosed thereof. In one embodiment, the host cell is a prokaryotic cell or a eukaryotic cell.

In a further aspect, the application provides CAR-T or CAR-NK cell. In one embodiment, such cell expresses the chimeric antigen receptors or the chimeric antigen receptor complexes as disclosed thereof.

In a further aspect, the application provides mammalian cells. In one embodiment, the mammalian cell comprises the chimeric antigen receptor or the chimeric antigen receptor complex as disclosed thereof. In one embodiment, the mammalian cell comprises the biomolecule complex as disclosed thereof.

In a further aspect, the application provides methods for treating tumor in a subject. In one embodiment, the method comprises administering to the subject a sufficient amount of the CAR-T or CAR-NK cell or non-viral vectors as disclosed

In a further aspect, the application provides pharmaceutical compositions. In one embodiment, the pharmaceutical composition comprises a therapeutically effective amount of the vectors, non-viral vectors, CAR-T or CAR-NK cell, proteins, biomolecules, or biomolecule complexes as disclosed thereof. In one embodiment, the pharmaceutical composition further comprises a pharmaceutically acceptable vehicle.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 shows AI-CAR expression cassettes (A-C) in a non-viral DNA construct encoding a monospecific, dual or bispecific CARs targeting CD19, CD22 and/or CD20 that are also capable of inducing the expression of proteins encoded within the AI-CAR vector that support CAR cell persistence and additional mechanisms of anti-tumor activity;

FIG. 2 depicts in vitro transcribed mRNAs that encode monospecific, dual or bispecific CARs targeting CD19, CD22 and/or CD20 as well an additional therapeutic protein with anti-tumor activity;

FIG. 3 depicts the mechanism of dual and bispecific AI-CAR targeting CD19 and CD22 or CD20 in a non-viral vector that induces the expression of genes such as cytokines and/or a bispecific antibody, encoded by the same integrated AI-CAR cassette;

FIG. 4 shows the result of a flow cytometry analysis of SW480 cells that express both CD19 and CD22 for specific binding of humanized anti-CD19scFv-Fc and anti-CD22scFv-Fc on day 1 post electroporation;

FIG. 5 shows the result of a flow cytometry analysis of CD19 and CD22 bispecific and dual specific CAR-T cell binding to recombinant human anti-CD19-Fc and anti-CD22-Fc. Dual specific CAR cells were generated by electroporating a plasmid mixure or as separate plasmids followed by pooling the monospecific CAR cells;

FIG. 6 shows a summary of dual and bispecific CAR expression on IL-15 expanded T cells, one day after electroporation (protein-L; A), and binding to rhCD19hFc (B), and rhCD22hFc (C);

FIG. 7 displays the cytotoxicity of CAR-T cells, expressing either single, dual, or bispecific anti-CD19 and/or anti-CD22 CARs, as measured by the viability of SW480 colon cancer cellsexpressing CD19, CD22 or both, in a 24 hour T cell-dependent cellular cytotoxicity (TDCC) assay; and

FIG. 8 displays the cytotoxicity of CAR-T cells, expressing dual, or bispecific anti-CD19 and anti-CD22 CARs, as measured by the viability of colon cancer cells SW480 expressing CD19, CD22 or both in a 24 hour T cell-dependent cellular cytotoxicity (TDCC) assay.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The disclosure provides, among others, isolated antibodies, methods of making such antibodies, bispecific or multi-specific molecules, antibody-drug conjugates and/or immuno-conjugates composed from such antibodies or antigen binding fragments, pharmaceutical compositions containing the antibodies, bispecific or multi-specific molecules, antibody-drug conjugates and/or immuno-conjugates, the methods for making the molecules and compositions, and the methods for treating cancer using the molecules and compositions disclosed herein.

The term “antibody” is used in the broadest sense and specifically covers single monoclonal antibodies (including agonist and antagonist antibodies), antibody compositions with polyepitopic specificity, as well as antibody fragments (e.g., Fab, F(ab′)₂, and Fv), so long as they exhibit the desired biological activity. In some embodiments, the antibody may be monoclonal, polyclonal, chimeric, single chain, bispecific or bi-effective, simianized, human and humanized antibodies as well as active fragments thereof. Examples of active fragments of molecules that bind to known antigens include Fab, F(ab′)₂, scFv and Fv fragments, including the products of an Fab immunoglobulin expression library and epitope-binding fragments of any of the antibodies and fragments mentioned above. In some embodiments, antibody may include immunoglobulin molecules and immunologically active portions of immunoglobulin molecules, i.e. molecules that contain a binding site that immunospecifically bind an antigen. The immunoglobulin can be of any type (IgG, IgM, IgD, IgE, IgA and IgY) or class (IgG1, IgG2, IgG3, IgG4, IgA1 and IgA2) or subclasses of immunoglobulin molecule. In one embodiment, the antibody may be whole antibodies and any antigen-binding fragment derived from the whole antibodies. A typical antibody refers to heterotetrameric protein comprising typically of two heavy (H) chains and two light (L) chains. Each heavy chain is comprised of a heavy chain variable domain (abbreviated as VH) and a heavy chain constant domain. Each light chain is comprised of a light chain variable domain (abbreviated as VL) and a light chain constant domain. The VH and VL regions can be further subdivided into domains of hypervariable complementarity determining regions (CDR), and more conserved regions called framework regions (FR). Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the light and heavy chains there are binding regions that interacts with the antigen.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by the hybridoma method first described by Kohler & Milstein, Nature, 256:495 (1975), or may be made by recombinant DNA methods (see, e.g., U.S. Pat. No. 4,816,567).

The monoclonal antibodies may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 [1984]).

Monoclonal antibodies can be produced using various methods including mouse hybridoma or phage display (see Siegel. Transfus. Clin. Biol. 9:15-22 (2002) for a review) or from molecular cloning of antibodies directly from primary B cells (see Tiller. New Biotechnol. 28:453-7 (2011)).

The term “antigen- or epitope-binding portion or fragment” refers to fragments of an antibody that are capable of binding to an antigen (herein, CD19, CD20, and CD22). These fragments may be capable of the antigen-binding function and additional functions of the intact antibody. Examples of binding fragments include, but are not limited to a single-chain Fv fragment (scFv) consisting of the VL and VH domains of a single arm of an antibody connected in a single polypeptide chain by a synthetic linker or a Fab fragment which is a monovalent fragment consisting of the VL, constant light (CL), VH and constant heavy 1 (CH1) domains. Antibody fragments can be even smaller sub-fragments and can consist of domains as small as a single CDR domain, in particular the CDR3 regions from either the VL and/or VH domains (for example see Beiboer et al., J. Mol. Biol. 296:833-49 (2000)). Antibody fragments are produced using conventional methods known to those skilled in the art. The antibody fragments are can be screened for utility using the same techniques employed with intact antibodies.

The “antigen-or epitope-binding fragments” can be derived from an antibody of the present disclosure by a number of art-known techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′)₂ fragment that has two antigen combining sites and is still capable of cross-linking antigen.

The Fab fragment may contain the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other, chemical couplings of antibody fragments are also known.

“Fv” is the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The “light chains” of antibodies (immunoglobulins) from any vertebrate species can be assigned to one of two clearly distinct types, called kappa and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called α, delta, epsilon, γ, and µ, respectively. The subunit structures and three-dimensional configurations of different classes of immunoglobulins are well known.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity. Methods to obtain “humanized antibodies” are well known to those skilled in the art. (see, e.g., Queen et al., Proc. Natl Acad Sci USA, 86:10029-10032 (1989), Hodgson et al., Bio/Technology, 9:421 (1991)).

The terms “polypeptide”, “peptide”, and “protein”, as used herein, are interchangeable and are defined to mean a biomolecule composed of amino acids linked by a peptide bond.

The terms “a”, “an” and “the” as used herein are defined to mean “one or more” and include the plural unless the context is inappropriate.

By “isolated” is meant a biological molecule free from at least some of the components with which it naturally occurs. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic a binding specificity.

“Recombinant” means the antibodies are generated using recombinant nucleic acid techniques in exogeneous host cells.

The term “antigen” refers to an entity or fragment thereof which can induce an immune response in an organism, particularly an animal, more particularly a mammal including a human. The term includes immunogens and regions thereof responsible for antigenicity or antigenic determinants.

Also, as used herein, the term “immunogenic” refers to substances which elicit or enhance the production of antibodies, T-cells or other reactive immune cells directed against an immunogenic agent and contribute to an immune response in humans or animals. An immune response occurs when an individual produces sufficient antibodies, T-cells and other reactive immune cells against administered immunogenic compositions of the present disclosure to moderate or alleviate the disorder to be treated.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

“Homology” between two sequences is determined by sequence identity. If two sequences which are to be compared with each other differ in length, sequence identity preferably relates to the percentage of the nucleotide residues of the shorter sequence which are identical with the nucleotide residues of the longer sequence. Sequence identity can be determined conventionally with the use of computer programs. The deviations appearing in the comparison between a given sequence and the above-described sequences of the disclosure may be caused for instance by addition, deletion, substitution, insertion or recombination.

The present disclosure may be understood more readily by reference to the following detailed description of specific embodiments and examples included herein. Although the present disclosure has been described with reference to specific details of certain embodiments thereof, it is not intended that such details should be regarded as limitations upon the scope of the disclosure.

To address the issues of cytokine signaling pathways in CAR expansion, terminal differentiation, and exhaustion, this application discloses the composition and method of using Artificial Immunesurveillance Chimeric Antigen Receptor (AI-CAR) for regulating the host response to CAR. The advancement of this AI-CAR technology aims to replace standard CAR manufacturing and enable an effective point of care therapy. While other CAR technologies may require the expression of soluble cytokine growth factors and/or multiple dosing for persistent activity to mount a complete and durable response, Al-CAR enables the production of CAR cells in the absence of either a constitutively active driver for proliferation or multiple CAR dosing for a durable anti-tumor response.

As compared to standard CAR, AI-CAR increases the efficiency of manufacturing CAR cells. AI-CAR may only require one target antigen for full proliferation and cytotoxic activity both in vitro and in vivo. In this context, AI-CAR may enable substantial reduction in manufacturing costs since the expansion of standard CAR-T cells generally requires the use of a combination of growth factors and aAPC for manufacturing.

Subsequently following AI-CAR engagement of tumor cells in vivo, the expression of several anti-tumor genes that are encoded by the integrated AI-CAR vector may be induced. The expression of these endogenous genes may enable patients to mount an anti-tumor response that more broadly targets different tumor antigens, such as neoantigens. For example, a STAT5 reporter system is used to induce STAT5 responsive genes in human T cells (Kanai, et al., 2014; Zeng, et al., 2016; Bednorz et al., 2011; and Fang et al., 2008). This feature is unique because standard CAR constructs typically are not capable of inducible gene expression. Together with these co-factors, Al-CAR will become a platform technology providing practical, economic, and effective solutions for the point of care cancer treatment.

Many forms of cancer may exist in a tumor environment that is immunosuppressive. AI-CAR will be highly desirable because AI-CAR vectors are designed to express additional anticancer genes that can decrease tumor immunosuppression and activate the patient’s anti-tumor immune response. One of the unique features of AI-CAR is its ability to regulate the expression of relevant anti-tumor genes at a tumor site and not to have them constitutively expressed. Another characteristic feature is that AI-CAR is designed to have a single dose at administration followed by its long-term activity and greater efficacy. With these advantageous features, Al-CAR is a better solution for the unmet challenge in the market, which promises the efficacy for treating most if not all types of cancer.

As to CAR therapeutics for treating hematologic cancers, there are multiple targets, such as CD19, CD20 and CD22, CD9, and CD38. The targeting strategy may involve the use of dual, bispecific AI-CAR encoded in either non-viral DNA vectors or transiently expressed RNA-CAR. In this way, AI-CAR may be used for greater persistence or as a transient treatment bridge to transplant. Either maybe used as a point a care treatment to induce a host anti-tumor immune response targeting neoantigens. Fewer DNA vector AI-CAR cells may be administered due to a robust in vivo expansion. In this context, a non-viral cassette construct encoding one or two CARs along with multiple inducible genes is a novel approach for a point of care treatment and to stimulate a durable host anti-tumor response. This configuration of CAR enables the production of CAR in T, NK, and other immune cells in the absence of either a potentially toxic constitutively active driver for proliferation or inflammatory cytokine, and for multiple CAR dosing, for a durable anti-tumor response. The bispecific CARs in this application not only target both CD19 and CD22 to prevent tumor escape via antigen loss but enables an induced response, such as cytokine, chemokine or a bispecific antibody, that is localized or restricted to tumor cell engagement.

### EXAMPLES

### Example 1. AI-CAR Constructs

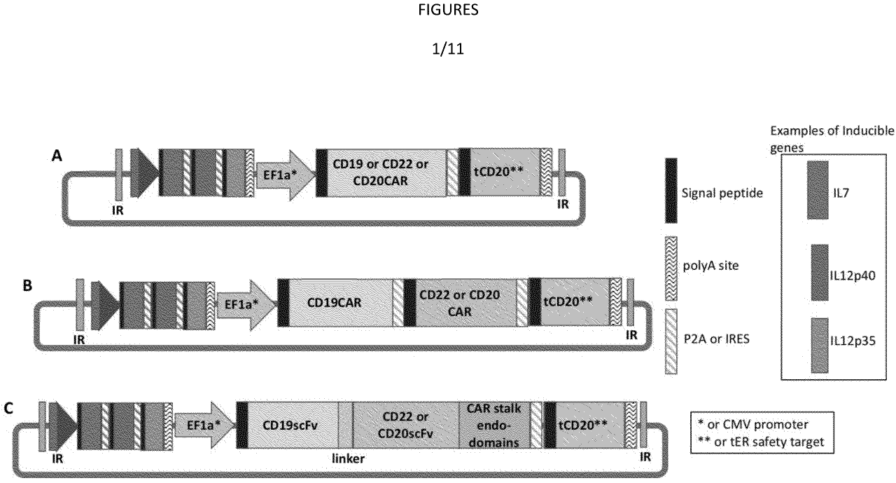

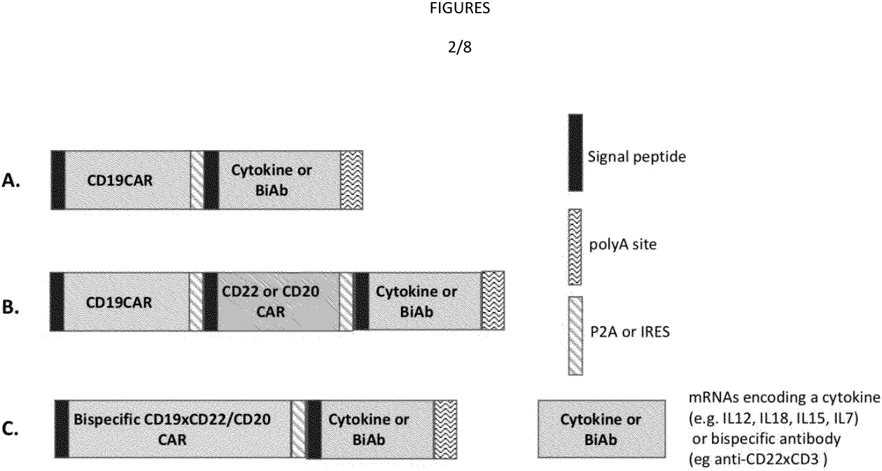

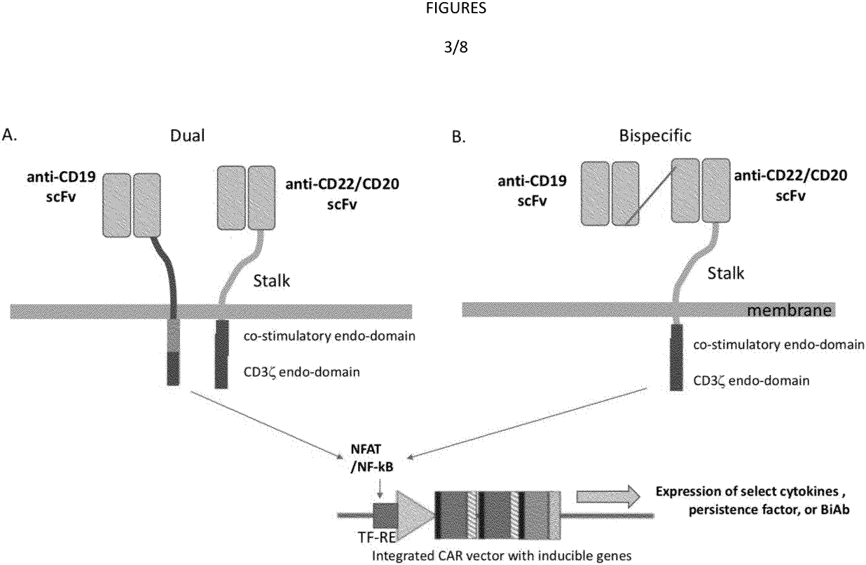

AI-CAR vectors with an inducible promoter may be constructed to induce the expression of additional proteins, following tumor engagement, with differnt anti-tumor mechanisms of activity to enhance CAR actiity. Thus AI-CAR constitute a combination’ therapy. A monospecific AI-CAR may be constructed by fusing a signal peptide, an anti-CD19 CAR scFv, a CD8 ectodomain stalk and transmembrane domain, a CD137 and a CD3z endodomain to a P2A ribosomal skip sequence or IRES followed by a signal peptide, a target for CAR elimination, e.g. a truncated EGFR or truncated CD20, and a polyA signal (FIG. 1A, SEQ ID1-5). To generate dual specific AI-CARs a second complete CAR sequence may be inserted between the first CAR and the safety target as indicated in FIG. 1B; SEQ ID 8. A bispecific AI-CAR may be generated by fusing the coding region of a signal peptide to a CD19 binding scFv and linker peptide, a CD22 or CD20 binding scFv, a CD8 hinge and transmembrane domain, and CD137 and CD3z endomains followed by a IRES or P2A sequence signal sequence safety target sequence and a pol A signal sequence (FIG. 1C, SEQ ID6 and 7). In addition, the AI-CAR vector contains an inducible promoter with transcription factor response elements (TF-RE) for transcription factors such as STAT, NFAT or NF-κB followed by the coding region for one or more genes (for example SEQ ID 9, 10, 16, 17) linked by an IRES or self-cleaving ribosomal skip peptide, such as T2A or P2A, followed by a polyA signal sequence as indicated in FIGS. 2A-C. Both coding regions may be located between transposon or viral terminal repeats (IR) for integration into T or NK cells. Alternatively, the coding regions of an Al-CAR construct may be integrated at a specific genomic site using zinc finger, TALEN or CRISPR/Cas9 nuclease (Eyquem 2017).

### Example 2. mRNA CAR for Transient Expression of Additional Mechanisms of Anti-Tumor Activity

Alternatively, Al-CARs with transient expression of additional mechanisms of anti-tumor activity may be expressed from in vitro transcribed (IVT) RNAs (FIGS. 2A-C, SEQ ID 11-15). The coding region for monospecific, dual or bispecific CARs are constructed as in FIG. 1. The mRNA Al-CARs are transeintly expressed and therefore do not require a target for CAR cell ellimination. The transiently expressed protein for addtional anti-tumor activity is included by the 3′ fusion of an IRES or P2A sequence, the coding region for a eg cytokine or bispecific antibody (SEQ ID 11-15) followed by a poly A tail. The mRNA AI-CAR is constructed within a vector for in vitro transcription (IVT). Thus the coding region is flanked by a T7 promoter and a polyA tail. The mRNA is transcribed with T7 polymerase and purified using standard methods and electroporated into T or NK cells. Additional proteins may also be co-expressed to enhance anti-tumor activity by co-transfecting their mRNA.

### Example 3. Examples of Al-CAR Designs

CAR T cells having an undifferentiated memory phenotype are characterized by in vivo persistence and the greatest therapeutic potential. To selectively expand the CAR cells with this phenotype and prevent terminal differentiation, several cytokines have been utilized, including IL15, IL7 and IL21. These cytokines also can promote T cell rejection of lymphoma (Markley 2010). Other proinflammatory cytokines may enhance the efficacy of CAR cells to eradicate tumors such as IL18 or IL12 (Chmielewski 2017, Kueberuwa 2018). Incorporating a combination of these cytokines into a CAR vector with expression regulated by CAR binding to tumor cells has several advantages including ease of CAR cell manufacturing (i.e. expansion in vitro), enabling point of care treatment, enhancing the activity and persistence of the CAR cell and potentially inducing or enhancing a patient anti-tumor response.

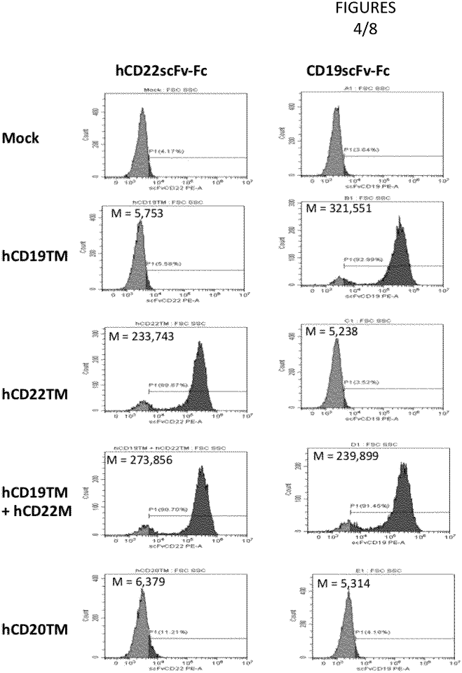

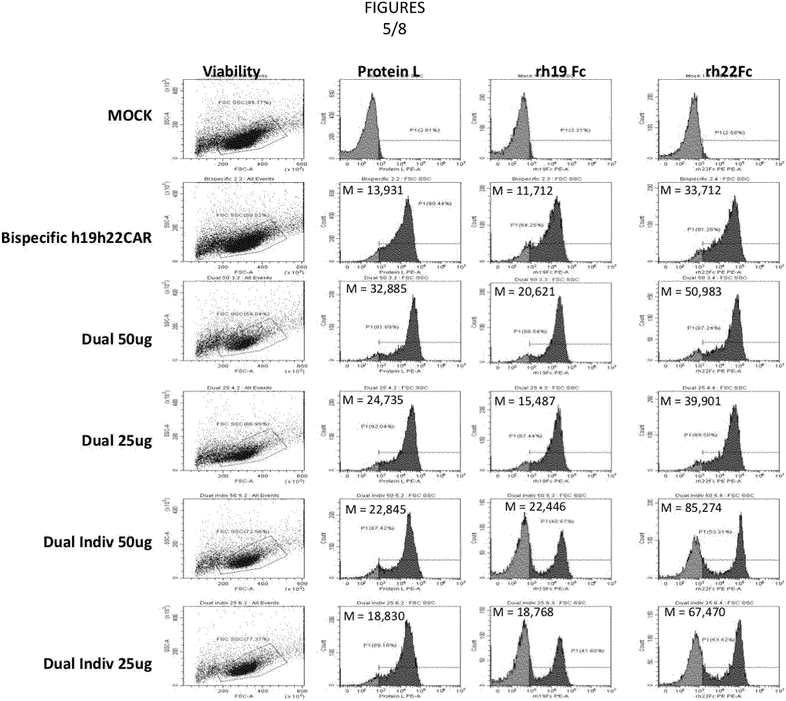

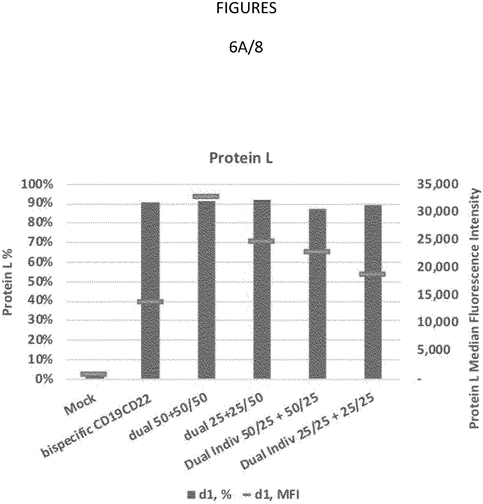

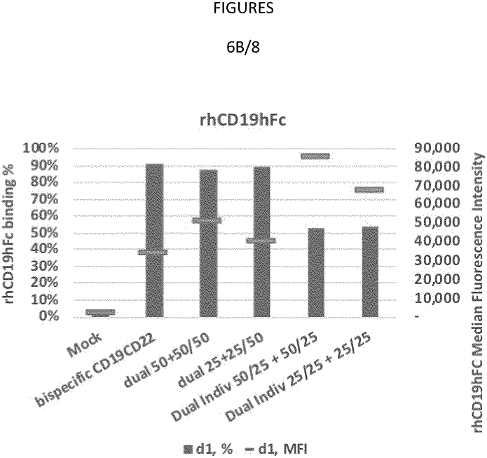

Two classes of CAR targeting CD19 and CD22 or CD20 were designed as shown in FIG. 3. A dual specific CAR complex comprises two independently configured receptors targeting CD19 and CD22, respectively (FIG. 3A). A bispecific CAR may be a classic second-generation CAR but contain two scFvs for binding to CD19 and CD22 or CD20 (FIG. 3B).

Subequent to binding tumor target antigens, the Al-CARs can activate transcription factor binding to the intergrated CAR vector inducible promoter to induce expression of genes that support AI-CAR cell persistence, undifferentiated memory phenotype and efficacy through additional anti-tumor mechansims (SEQ ID 9, 10, 16, 17). For example, these activties may be provided by certain cytokines and through a cytototoxic bispecific antibody such as an anti-CD20xNKG2D to target NK cytotoxicity to tumor cells. Transient expression of additonal cytotoxic mechanisms decreases the risk of for example off-tumor cytotoxicity relative to them being constituitively expressed. Alternatively the additional mechanisms of anti-tumor activity can also be transiently expressed using mRNA AI-CARs (SEQ ID 11-17).

### Example 4. Humanized anti-CD19scFv-Fc and anti-CD22scFv-Fc Specifically Bind to Target Antigens

To demonstrate fuction of humanized anti-CD19scFv and anti-CD22scFv used for AI-CAR construction, the scFvs were fused to Fc and expressed in HEK-293 cells. The anti-CD19 and anti-CD22 scFv bound specifically to SW480 cells that had been transfected with CD19 or CD20 mRNA. The scFvs did not bind to mock transfectants or cells transfected with a CD20 mRNA. Binding was determined 1 day after SW480 electroporation with the CD19, CD22 and CD20 mRNAs. Binding was determined by standard flow cytofluorimetry.

### Example 5. CD19 and CD22 Binding to CAR T Cells

Humanized dual and bispecific CARs were expressed in T cells to demonstrate their binding to CD19 and CD22. T cells were electroporated with mRNA encoding the different CARs. A combination of CD19 and CD22 CAR RNAs were co-electroporated into T cells using a total of 50 or 100ug of RNA per 50×10^6 cells (Dual). CD19 and CD22 CAR RNAs were also individually electroporated into T cells using 25 or 50 µg of each RNA per 50×10^6 cells. The individual or monospecific CARs cells were then pooled (Dual individual). One day after electroporation CAR expression was demonstrated by the binding of protein-L as determined by standard flow cytometry. Soluble recombinant CD19-Fc and CD22-Fc bound specifically to the dual and bispecific CAR and not to mock transfected T cells. The data indicates that a greater percent of dual or co-electroporated T cells bind CD19 and CD22 relative to individually electroporated and pooled CAR T cells.

### Example 6. Summary of CAR Expression on IL-15 Expanded T Cells Day 1 Post Electroporation

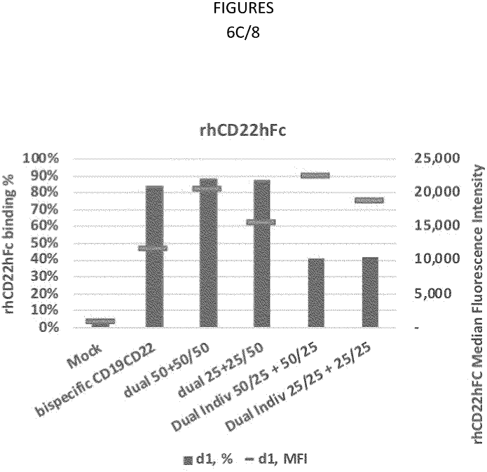

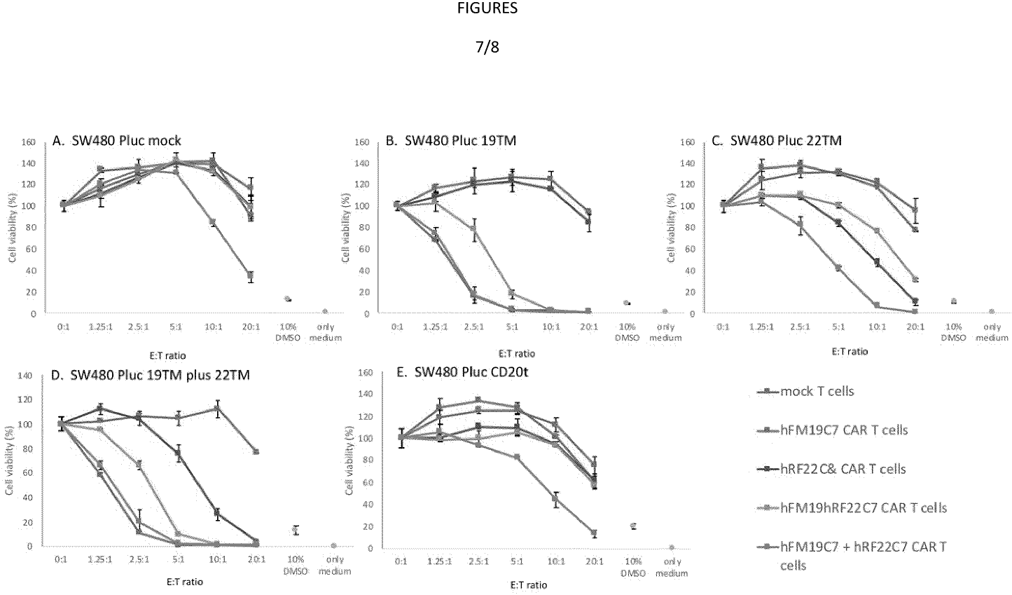

FIG. 6 summarizes the percent and level (median fluorescence intensity) of CD19-Fc and CD22-Fc binding to T cells expressing the humanized CD19 and CD22 RNA CARS one day post electroporation. T cells were electroporated with different quantities of CAR RNA as indicated. CAR expression was demonstrated by the binding of protein-L as determined by standard flow cytometry.

### Example 7. Monospecific, Dual and Bispecific CD19 and CD22 CAR T Cell Cytotoxicity

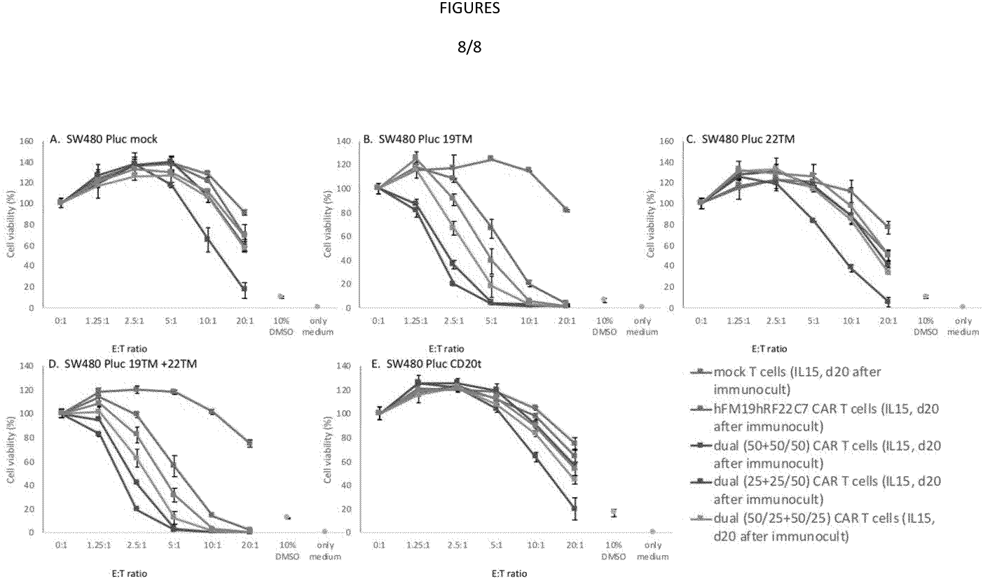

The cytotoxic activity of monospecific, dual and bispecific CD19 and CD22 CAR cells was determined. T cells were electroporated with CAR RNAs and SW480 expressing luciferase were electroporated with transmembrane forms of CD19, CD22 or both. CD20 was also expressed in SW480 as a negative control. One day later CAR T cells were co-incubated with target cells over a range of effector to target ratios from 0:1 to 20:1. Cytotoxicity was determined at 24 hours by determining residual luciferase activity using a multimode plate reader. The monospecific CD19 CAR demonstrated the most potent killing followed by the dual CD19, CD22 CAR cells. However, the dual CD19, CD22 CAR cells demonstrated the greatest background killing of mock and CD20 expressing targets. This non-specific activity may reflect the higher level of input RNA in the in the co-electroporation which was 2x the quantity (100 µg total per 50 ×10^6 T cells) used for the other electroporations. This was address in a subsequent cytotoxicity assay (FIG. 8).

### Example 8. Dual and Bispecific CD19 and CD22 CAR T Cell Cytotoxicity

The cytotoxic activity of dual and bispecific CD19 and CD22 CAR cells was determined. T cells were electroporated with CAR RNAs and SW480 expressing luciferase were electroporated with transmembrane forms of CD19, CD22 or both. CD20 was also expressed as a negative control. One day later CAR T cells were co-incubated with target cells over a range of effector to target ratios from 0:1 to 20:1. Cytotoxicity was determined at 24 hours by determining residual luciferase activity using a multimode plate reader. The data indicates that dual CAR co-electroporated with 25 ug of CD19 RNA and 25 ug of CD22 RNA (per 50×10^6 cells) has greater specific activity relative to dual CAR electroporated with 2x more RNA. In addition, the dual CAR format demonstrated greater activity relative to the bispecific CAR.

### SEQUENCE LISTING

1. Humanized CD19 CAR sequences. The humanized CD19 scFvs, derived from FMC63 mouse antibodies (Nicholson 1997), was fused to a CD8 stalk/hinge, CD8 transmembrane domain, CD137 endo-domain and CD3ζ endo-domain. A safety target is fused using a P2A peptide.

SEQ ID NO:1h1FM19C7tERMKHLWFFLLLVAAPRWVLSQVQLQESGP
GLVKPSETLSLTCTVSGGSIPDYGVSWIRQPPGKGLEWIGVIWGSETTYY
NSALKSRVTISVDTSKNQFSLKLSSVTAADTAVYYCARHYYYGGSYAMDY
WGQGTSLTVSSGGGGSGGGGSGGGSGDIQMTQSPSSLSASVGDRVTITCR
ASQDISKYLNWYQQKPGKAPKLLIYHTSRLHSGVPSRFSGSGSGTDFTLT
ISSLQPEDFATYYCQQGNTLPYTFGGGTKVEITGAPTTTPAPRPPTPAPT
IASQPLSLRPEACRPAAGGAVHTRGLDFACDIYIWAPLAGTCGVLLLSLV
ITLYCKRGRKKLLYIFKQPFMRPVQTTQEEDGCSCRFPEEEEGGCELRVK
FSRSADAPAYQQGQNQLYNELNLGRREEYDVLDKRRGRDPEMGGKPQRRK
NPQEGLYNELQKDKMAEAYSEIGMKGERRRGKGHDGLYQGLSTATKDTYD
ALHMQALPPRASGSGATNFSLLKQAGDVEENPGP MNAKVVVVLVLVLTAL
CLSDGRKVCNGIGIGEFKDSLSINATNIKHFKNCTSISGDLHILPVAFRG
DSFTHTPPLDPQELDILKTVKEITGFLLIQAWPENRTDLHAFENLEllRG
RTKQHGQFSLAVVSLNITSLGLRSLKEISDGDVIISGNKNLCYANTINWK
KLFGTSGQKTKIISNRGENSCKATGQVCHALCSPEGCWGPEPRDCVSCRN
VSRGRECVDKCNLLEGEPREFVENSECIQCHPECLPQAMNITCTGRGPDN
CIQCAHYIDGPHCVKTCPAGVMGENNTLVWKYADAGHVCHLCHPNCTYGC
TGPGLEGCPTNGPKIPSIATGMVGALLLLLVVALGIGLFMRR

SEQ ID NO:2h2FM19C7tERMKHLWFFLLLVAAPRWVLSDIQMTQSPS
SLSASVGDRVTITCRASQDISKYLNWYQQKPGGAPKLLIYHTSRLHSGVP
SRFSGSGSGTDFTFTISSLQQEDIATYYCQQGNTLPYTFGGGTKVEIKGG
GGSGGGGSGGGGSQVQLQESGPGLVKPSQTLSLTCTVSGVSLPDYGVSWI
RQHPGKGLEWIGVIWGSETTYYNSALKSRVTISVDTSKNQFSLKLSSVTA
ADTAVYYCARHYYYGGSYAMDYWGQGTLVTVSSGAPTTTPAPRPPTPAPT
IASQPLSLRPEACRPAAGGAVHTRGLDFACDIYIWAPLAGTCGVLLLSLV
ITLYCKRGRKKLLYIFKQPFMRPVQTTQEEDGCSCRFPEEEEGGCELRVK
FSRSADAPAYQQGQNQLYNELNLGRREEYDVLDKRRGRDPEMGGKPQRRK
NPQEGLYNELQKDKMAEAYSEIGMKGERRRGKGHDGLYQGLSTATKDTYD
ALHMQALPPRASGSGATNFSLLKQAGDVEENPGP MNAKVVVVLVLVLTAL
CLSDGRKVCNGIGIGEFKDSLSINATNIKHFKNCTSISGDLHILPVAFRG
DSFTHTPPLDPQELDILKTVKEITGFLLIQAWPENRTDLHAFENLEIIRG
RTKQHGQFSLAVVSLNITSLGLRSLKEISDGDVIISGNKNLCYANTINWK
KLFGTSGQKTKIISNRGENSCKATGQVCHALCSPEGCWGPEPRDCVSCRN
VSRGRECVDKCNLLEGEPREFVENSECIQCHPECLPQAMNITCTGRGPDN
CIQCAHYIDGPHCVKTCPAGVMGENNTLVWKYADAGHVCHLCHPNCTYGC
TGPGLEGCPTNGPKIPSIATGMVGALLLLLVVALGIGLFMRR

SEQ ID NO:3h3FM19C7tERMKHLWFFLLLVAAPRWVLSQVQLQESGP
GLVKPSQTLSLTCTVSGVSIPDYGVSWIRQHPGKGLEWIGVIWGSETTYY
NSALKSRVTISVDTSKNQFSLKLSSVTAADTAVYYCARHYYYGGSYAMDY
WGQGTLVTVSSGGGGSGGGGSGGGGSDIQMTQSPSSLSASVGDRVTITCR
ASQDISKYLNWYQQKPGKAPKLLIYHTSRLHSGVPSRFSGSGSGTDFTFT
ISSLQQEDIATYYCQQGNTLPYTFGGGTKVElKGAPTTTPAPRPPTPAPT
IASQPLSLRPEACRPAAGGAVHTRGLDFACDIYIWAPLAGTCGVLLLSLV
ITLYCKRGRKKLLYIFKQPFMRPVQTTQEEDGCSCRFPEEEEGGCELRVK
FSRSADAPAYQQGQNQLYNELNLGRREEYDVLDKRRGRDPEMGGKPQRRK
NPQEGLYNELQKDKMAEAYSEIGMKGERRRGKGHDGLYQGLSTATKDTYD
ALHMQALPPRASGSGATNFSLLKQAGDVEENPGP MNAKVVVVLVLVLTAL
CLSDGRKVCNGIGIGEFKDSLSINATNIKHFKNCTSISGDLHILPVAFRG
DSFTHTPPLDPQELDILKTVKEITGFLLIQAWPENRTDLHAFENLEIIRG
RTKQHGQFSLAVVSLNITSLGLRSLKEISDGDVIISGNKNLCYANTINWK
KLFGTSGQKTKIISNRGENSCKATGQVCHALCSPEGCWGPEPRDCVSCRN
VSRGRECVDKCNLLEGEPREFVENSECIQCHPECLPQAMNITCTGRGPDN
CIQCAHYIDGPHCVKTCPAGVMGENNTLVWKYADAGHVCHLCHPNCTYGC
TGPGLEGCPTNGPKIPSIATGMVGALLLLLVVALGIGLFMRR

2. Humanized CD22 CAR sequences. The humanized CD22 specific scFvs, derived from RFB4 mouse antibodies (Mansfeild 1997), was fused to a CD8 stalk/hinge, CD8 transmembrane domain, CD137 endo-domain and CD3ζ endo-domain.

SEQ ID NO:4h1RF22C7tERMKHLWFFLLLVAAPRWVLSEVQLVESGG
GLVQPGGSLRLSCAASGFAFSIYDMSWVRQAPGKGLEWVSYISSGGGTTY
YPDTVKGRFTISRDNAKNSLYLQMNSLRAEDTAVYYCARHSGYGSSYGVL
FAYWGQGTLVTVSGGGGSGGGGSGGGGSDIQMTQSPSSLSASVGDRVTIT
CRASQDISNYLNWYQQKPGKAPKLLIYYTSILHSGVPSRFSGSGSGTDFT
FTISSLQQEDIATYYCQQGNTLPWTFGGGTKVEIKGAPTTTPAPRPPTPA
PTIASQPLSLRPEACRPAAGGAVHTRGLDFACDIYIWAPLAGTCGVLLLS
LVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPQR
RKNPQEGLYNELQKDKMAEAYSEIGMKGERRRGKGHDGLYQGLSTATKDT
YDALHMQALPPRASGSGATNFSLLKQAGDVEENPGP MNAKVVVVLVLVLT
ALCLSDGRKVCNGIGIGEFKDSLSINATNIKHFKNCTSISGDLHILPVAF
RGDSFTHTPPLDPQELDILKTVKEITGFLLIQAWPENRTDLHAFENLEII
RGRTKQHGQFSLAVVSLNITSLGLRSLKEISDGDVIISGNKNLCYANTIN
WKKLFGTSGQKTKIISNRGENSCKATGQVCHALCSPEGCWGPEPRDCVSC
RNVSRGRECVDKCNLLEGEPREFVENSECIQCHPECLPQAMNITCTGRGP
DNCIQCAHYIDGPHCVKTCPAGVMGENNTLVWKYADAGHVCHLCHPNCTY
GCTGPGLEGCPTNGPKIPSIATGMVGALLLLLVVALGIGLFMRR

SEQ ID NO:5h2RF22C7tERMKHLWFFLLLVAAPRWVLSEVQLVESGG
GLVQPGGSLRLSCAASGFTFSIYDMSWVRQAPGKGLEWVSYISSGGGTTY
YPDTVKGRFTISRDNAKNSLYLQMNSLRAEDTAVYYCARHSGYGSSYGVL
FAYWGQGTLVTGGGGSGGGGSGGGGSDIQMTQSPSSLSASVGDRVTITCR
ASQDISNYLNWYQQKPGKAPKLLIYYTSILHSGVPSRFSGSGSGTDFTLT
ISSLQQEDFATYYCQQGNTLPWTFGGGTKVEIKGAPTTTPAPRPPTPAPT
IASQPLSLRPEACRPAAGGAVHTRGLDFACDIYIWAPLAGTCGVLLLSLV
ITLYCKRGRKKLLYIFKQPFMRPVQTTQEEDGCSCRFPEEEEGGCELRVK
FSRSADAPAYQQGQNQLYNELNLGRREEYDVLDKRRGRDPEMGGKPQRRK
NPQEGLYNELQKDKMAEAYSEIGMKGERRRGKGHDGLYQGLSTATKDTYD
ALHMQALPPRASGSGATNFSLLKQAGDVEENPGP MNAKVVVVLVLVLTAL
CLSDGRKVCNGIGIGEFKDSLSINATNIKHFKNCTSISGDLHILPVAFRG
DSFTHTPPLDPQELDILKTVKEITGFLLIQAWPENRTDLHAFENLEIIRG
RTKQHGQFSLAVVSLNITSLGLRSLKEISDGDVIISGNKNLCYANTINWK
KLFGTSGQKTKIISNRGENSCKATGQVCHALCSPEGCWGPEPRDCVSCRN
VSRGRECVDKCNLLEGEPREFVENSECIQCHPECLPQAMNITCTGRGPDN
CIQCAHYIDGPHCVKTCPAGVMGENNTLVWKYADAGHVCHLCHPNCTYGC
TGPGLEGCPTNGPKIPSIATGMVGALLLLLVVALGIGLFMRR

3. Humanized CD19 and CD22 bispecific CAR sequences. A bispecific CAR targeting CD19 and CD22 may be generated by the fusion a CD19 scFv (with leader) to a linker sequence followed by the CD22 scFv, a CD8 stalk/hinge, CD8 transmembrane domain, a CD137 endo-domain and a CD3? endo-domain.

SEQ ID NO:6hRC19hRF22C7tERMKHLWFFLLLVAAPRWVLSQVQLQ
ESGPGLVKPSQTLSLTCTVSGVSLPDYGVSWIRQHPGKGLEWIGVIWGSE
TTYYNSALKSRVTISVDTSKNQFSLKLSSVTAADTAVYYCARHYYYGGSY
AMDYWGQGTLVTVSSGGGGSGGGGSGGGGSDIQMTQSPSSLSASVGDRVT
ITCRASQDISKYLNWYQQKPGGAPKLLIYHTSRLHSGVPSRFSGSGSGTD
FTFTISSLQQEDIATYYCQQGNTLPYTFGGGTKVElKPAGGGEPKSSDKT HTGGASEVQLVESGGGLVQPGGSLRLSCAASGFAFSIYD MSWVRQAPGKG
LEWVSYISSGGGTTYYPDTVKGRFTISRDNAKNSLYLQMNSLRAEDTAVY
YCARHSGYGSSYGVLFAYWGQGTLVTVSGGGGSGGGGSGGGGSDIQMTQS
PSSLSASVGDRVTITCRASQDISNYLNWYQQKPGKAPKLLIYYTSILHSG
VPSRFSGSGSGTDFTFTISSLQQEDIATYYCQQGNTLPWTFGGGTKVEIK
GAPTTTPAPRPPTPAPTIASQPLSLRPEACRPAAGGAVHTRGLDFACDIY
IWAPLAGTCGVLLLSLVITLYCKRGRKKLLYIFKQPFMRPVQTTQEEDGC
SCRFPEEEEGGCELRVKFSRSADAPAYQQGQNQLYNELNLGRREEYDVLD
KRRGRDPEMGGKPQRRKNPQEGLYNELQKDKMAEAYSEIGMKGERRRGKG
HDGLYQGLSTATKDTYDALHMQALPPRASGSGATNFSLLKQAGDVEENPG P MNAKVVVVLVLVLTALCLSDGRKVCNGIGIGEFKDSLSINATNIKHFKN
CTSISGDLHILPVAFRGDSFTHTPPLDPQELDILKTVKEITGFLLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFMRR

SEQ ID NO:7h3FM19h2RF22C7tERMKHLWFFLLLVAAPRWVLSQVQ
LQESGPGLVKPSQTLSLTCTVSGVSIPDYGVSWIRQHPGKGLEWIGVIWG
SETTYYNSALKSRVTISVDTSKNQFSLKLSSVTAADTAVYYCARHYYYGG
SYAMDYWGQGTLVTVSSGGGGSGGGGSGGGGSDIQMTQSPSSLSASVGDR
VTITCRASQDISKYLNWYQQKPGKAPKLLIYHTSRLHSGVPSRFSGSGSG
TDFTFTISSLQQEDIATYYCQQGNTLPYTFGGGTKVEIKPAGGGEPKSSD KTHTGGASEVQLVESGGGLVQPGGSLRLSCAASGFTFSIYDMSWVRQAPG
KGLEWVSYISSGGGTTYYPDTVKGRFTISRDNAKNSLYLQMNSLRAEDTA
VYYCARHSGYGSSYGVLFAYWGQGTLVTGGGGSGGGGSGGGGSDIQMTQS
PSSLSASVGDRVTITCRASQDISNYLNWYQQKPGKAPKLLIYYTSILHSG
VPSRFSGSGSGTDFTLTISSLQQEDFATYYCQQGNTLPWTFGGGTKVEIK GAPTTTPAPRPPTPAPTIASQPLSLRPEACRPAAGGAVHTRGLDFACDIY
IWAPLAGTCGVLLLSLVITLYCKRGRKKLLYI FKQPFMRPVQTTQEEDG
CSCRFPEEEEGGCELRVKFSRSADAPAYQQGQNQLYNELNLGRREEYDVL
DKRRGRDPEMGGKPQRRKNPQEGLYNELQKDKMAEAYSEIGMKGERRRGK
GHDGLYQGLSTATKDTYDALHMQALPPRASGSGATNFSLLKQAGDVEENP GP MNAKVVVVLVLVLTALCLSDGRKVCNGIGIGEFKDSLSINATNIKHFK
NCTSISGDLHILPVAFRGDSFTHTPPLDPQELDILKTVKEITGFLLIQAW
PENRTDLHAFENLEIIRGRTKQHGQFSLAVVSLNITSLGLRSLKEISDGD
VIISGNKNLCYANTINWKKLFGTSGQKTKIISNRGENSCKATGQVCHALC
SPEGCWGPEPRDCVSCRNVSRGRECVDKCNLLEGEPREFVENSECIQCHP
ECLPQAMNITCTGRGPDNCIQCAHYIDGPHCVKTCPAGVMGENNTLVWKY
ADAGHVCHLCHPNCTYGCTGPGLEGCPTNGPKIPSIATGMVGALLLLLVV
ALGIGLFMRR

4. Humanized CD19 and CD22 dual CAR sequences. A dual CAR targeting CD19 and CD22 may be generated by the fusion a CD19 CAR (with leader) to a ribosomal skip peptide, such as T2A or P2A followed by a CD22 CAR.

SEQ ID NO:8h2FM19C7xh2RF22C7tERMKHLWFFLLLVAAPRWVLS
QVQLQESGPGLVKPSQTLSLTCTVSGVSLPDYGVSWIRQHPGKGLEWIGV
IWGSETTYYNSALKSRVTISVDTSKNQFSLKLSSVTAADTAVYYCARHYY
YGGSYAMDYWGQGTLVTVSSGGGGSGGGGSGGGGSDIQMTQSPSSLSASV
GDRVTITCRASQDISKYLNWYQQKPGGAPKLLIYHTSRLHSGVPSRFSGS
GSGTDFTFTISSLQQEDIATYYCQQGNTLPYTFGGGTKVEIKGAPTTTPA
PRPPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEE
EGGCELRVKFSRSADAPAYQQGQNQLYNELNLGRREEYDVLDKRRGRDPE
MGGKPQRRKNPQEGLYNELQKDKMAEAYSEIGMKGERRRGKGHDGLYQGL
STATKDTYDALHMQALPPRGSGATNFSLLKQAGDVEENPGPEVQLVESGG
GLVQPGGSLRLSCAASGFTFSIYDMSWVRQAPGKGLEWVSYISSGGGTTY
YPDTVKGRFTISRDNAKNSLYLQMNSLRAEDTAVYYCARHSGYGSSYGVL
FAYWGQGTLVTGGGGSGGGGSGGGGSDIQMTQSPSSLSASVGDRVTITCR
ASQDISNYLNWYQQKPGKAPKLLIYYTSILHSGVPSRFSGSGSGTDFTLT
ISSLQQEDFATYYCQQGNTLPWTFGGGTKVEIKGAPTTTPAPRPPTPAPT
IASQPLSLRPEACRPAAGGAVHTRGLDFACDIYIWAPLAGTCGVLLLSLV
ITLYCKRGRKKLLYIFKQPFMRPVQTTQEEDGCSCRFPEEEEGGCELRVK
FSRSADAPAYQQGQNQLYNELNLGRREEYDVLDKRRGRDPEMGGKPQRRK
NPQEGLYNELQKDKMAEAYSEIGMKGERRRGKGHDGLYQGLSTATKDTYD
ALHMQALPPRASGSGATNFSLLKQAGDVEENPGP MNAKVVVVLVLVLTAL
CLSDGRKVCNGIGIGEFKDSLSINATNIKHFKNCTSISGDLHILPVAFRG
DSFTHTPPLDPQELDILKTVKEITGFLLIQAWPENRTDLHAFENLEIIRG
RTKQHGQFSLAVVSLNITSLGLRSLKEISDGDVIISGNKNLCYANTINWK
KLFGTSGQKTKIISNRGENSCKATGQVCHALCSPEGCWGPEPRDCVSCRN
VSRGRECVDKCNLLEGEPREFVENSECIQCHPECLPQAMNITCTGRGPDN
CIQCAHYIDGPHCVKTCPAGVMGENNTLVWKYADAGHVCHLCHPNCTYGC
TGPGLEGCPTNGPKIPSIATGMVGALLLLLVVALGIGLFMRR

5. Examples of combinatorial expression of cytokines in CAR cells to enhance anti-tumor activity. A combination of IL7, IL15 or IL21 may be transiently expressed in CAR cells to activate CAR cells or host T and NK cells to induce proliferation and anti-tumor cytotoxicity. A combination sequence of IL7, IL15 and IL21 may consist of a signal peptide, a cytokine, a ribosomal skip peptide, such a P2A and another cytokine.

SEQ ID NO:9IL7 x IL21MKHLWFFLLLVAAPRWVLSDCDIEGKDGK
QYESVLMVSIDQLLDSMKEIGSNCLNNEFNFFKRHICDANKEGMFLFRAA
RKLRQFLKMNSTGDFDLHLLKVSEGTTILLNCTGQVKGRKPAALGEAQPT
KSLEENKSLKEQKKLNDLCFLKRLLQEIKTCWNKILMGTKEHPAG GGTKT
ESSSRGQGQDRHMIRMRQLIDIVDQLKNYVNDVPEFLPAPEDVETNCEWS
AFSCFQKAQLKSANTGNNERIINVSIKKLKRKPPSTNAGRRQKHRLTCPS
CDSYEKKPPKEFLERFKSLLQKMIHQHLSSRTHGSEDS

SEQ ID NO:10IL7 x IL15MKHLWFFLLLVAAPRWVLSDCDIEGKDG
KQYESVLMVSIDQLLDSMKEIGSNCLNNEFNFFKRHICDANKEGMFLFRA
ARKLRQFLKMNSTGDFDLHLLKVSEGTTILLNCTGQVKGRKPAALGEAQP
TKSLEENKSLKEQKKLNDLCFLKRLLQEIKTCWNKILMGTKEHPAG GGTK
TESSSRGGIHVFILGCFSAGLPKTEANWVNVISDLKKIEDLIQSMHIDAT
LYTESDVHPSCKVTAMKCFLLELQVISLESGDASIHDTVENLIILANNSL
SSNGNVTESGCKECEELEEKNIKEFLQSFVHIVQMFINTS

### 6. Examples of RNA CARs

SEQ ID NO:11h5RM19C7-P2A-h2RF22U1 (Figure 2A)MKHLW
FFLLLVAAPRWVLSQVQLQESGPGLVKPSQTLSVTCTVSGVSLPDYGVSW
IRQPPRKGLEWLGVIWGSETTYYNSALKSRLTISKDNSKSQVSLKMSSVT
AADTAVYYCAKHYYYGGSYAMDYWGQGTLVTVSSGGGGSGGGGSGGGGSD
IQQMTAQSPSSLSASVGDRVTITCRASQDISKYLNWYQQKPDGTVKLLIY
HTSRLHSGVPSRFSGSGSGTDYSLTISSLQPEDIATYFCQQGNTLPYTFG
GGTKVEIKGAP TTTPAPRPPTPAPTIASQPLSLRPEACRPAAGGAVHTRG
LDFACDIYIWAPLAGTCGVLLLSLVITLYCKRGRKKLLYIFKQPFMRPVQ
TTQEEDGCSCRFPEEEEGGCELRVKFSRSADAPAYQQGQNQLYNELNLGR
REEYDVLDKRRGRDPEMGGKPQRRKNPQEGLYNELQKDKMAEAYSEIGMK
GERRRGKGHDGLYQGLSTATKDTYDALHMQALPPRAS GSGATNFSLLKQA
GDVEENPGP MEFGLSWVFLVALLRGVQCEVQLESGGGLVQPGGSLRLSCA
ASGFTFSIYDMSWVRQAPGKGLEWVSYISSGGGTTYYPDTVKGRFTISRD
NAKNSLYLQMNSLRAEDTAVYYCARHSGYGSSYGVLFAYWGQGTLVTGGG
GSGGGGSGGGGSDIQMTQSPSSLSASVGDRVTITCRASQDISNYLNWYQQ
KPGKAPKLLIYYTSILHSGVPSRFSGSGSGTDFTLTISSLQQEDFATYYC
QQGNTLPWTFGGGTKVEIKPAGGGGGSEVQLVESGGGLVQPGGSLRLSCA
ASGYSFTGYTMNWVRQAPGKGLEWVALINPYKGVSTYNQKFKDRFTISVD
KSKNTAYLQMNSLRAEDTAVYYCARSGYYGDSDWYFDVWGQGTLVTVSSG
GGGSGGGGSGGGGSDIQMTQSPSSLSASVGDRVTITCRASQDIRNYLNWY
QQKPGKAPKLLIYYTSRLESGVPSRFSGSGSGTDYTLTISSLQPEDFATY
YCQQGNTLPWTFGQGTKVEIKP

SEQ ID NO:12h5RM19C7-P2A-IL18 (Figure 2A)MKHLWFFLL
LVAAPRWVLSQVQLQESGPGLVKPSQTLSVTCTVSGVSLPDYGVSWIRQP
PRKGLEWLGVIWGSETTYYNSALKSRLTISKDNSKSQVSLKMSSVTAADT
AVYYCAKHYYYGGSYAMDYWGQGTLVTVSSGGGGSGGGGSGGGGSDIQQM
TAQSPSSLSASVGDRVTITCRASQDISKYLNWYQQKPDGTVKLLIYHTSR
LHSGVPSRFSGSGSGTDYSLTISSLQPEDIATYFCQQGNTLPYTFGGGTK
VEIKGAP TTTPAPRPPTPAPTIASQPLSLRPEACRPAAGGAVHTRGLDFA
CDIYIWAPLAGTCGVLLLSLVITLYCKRGRKKLLYIFKQPFMRPVQTTQE
EDGCSCRFPEEEEGGCELRVKFSRSADAPAYQQGQNQLYNELNLGRREEY
DVLDKRRGRDPEMGGKPQRRKNPQEGLYNELQKDKMAEAYSEIGMKGERR
RGKGHDGLYQGLSTATKDTYDALHMQALPPRAS GSGATNFSLLKQAGDVE
ENPGP MEFGLSWVFLVALLRGVQCYFGKLESKLSVIRNLNDQVLFIDQGN
RPLFEDMTDSDCRDNAPRTIFIISMYKDSQPRGMAVTISVKCEKISTLSC
ENKIISFKEMNPPDNIKDTKSDIIFFQRSVPGHDNKMQFESSSYEGYFLA
CEKERDLFKLILKKEDELGDRSIMFTVQNED

SEQ ID NO:13h5RM19C7-P2A-h2RF22C7-P2A-IL18 (Figure 2B)MKHLWFFLLLVAAPRWVLSQVQLQESGPGLVKPSQTLSVTCTVSGV
SLPDYGVSWIRQPPRKGLEWLGVIWGSETTYYNSALKSRLTISKDNSKSQ
VSLKMSSVTAADTAVYYCAKHYYYGGSYAMDYWGQGTLVTVSSGGGGSGG
GGSGGGGSDIQQMTAQSPSSLSASVGDRVTITCRASQDISKYLNWYQQKP
DGTVKLLIYHTSRLHSGVPSRFSGSGSGTDYSLTISSLQPEDIATYFCQQ GNTLPYTFGGGTKVEIKGAP TTTPAPRPPTPAPTIASQPLSLRPEACRPA
AGGAVHTRGLDFACDIYIWAPLAGTCGVLLLSLVITLYCKRGRKKLLYIF
KQPFMRPVQTTQEEDGCSCRFPEEEEGGCELRVKFSRSADAPAYQQGQNQ
LYNELNLGRREEYDVLDKRRGRDPEMGGKPQRRKNPQEGLYNELQDKMAE
AYSEIGMKGERRRGKGHDGLYQGLSTATKDTYDALHMQALPPRAS GSGAT
NFSLLKQAGDVEENPGP MKHLWFFLLLVAAPRWVLSEVQLVESGGGLVQP
GGSLRLSCAASGFTFSIYDMSWVRQAPGKGLEWVSYISSGGGTTYYPDTV
KGRFTISRDNAKNSLYLQMNSLRAEDTAVYYCARHSGYGSSYGVLFAYWG
QGTLVTGGGGSGGGGSGGGGSDIQMTQSPSSLSASVGDRVTITCRASQDI
SNYLNWYQQKPGKAPKLLIYYTSILHSGVPSRFSGSGSGTDFTLTISSLQ
QEDFATYYCQQGNTLPWTFGGGTKVEIKPAGTTTPAPRPPTPAPTIASQP
LSLRPEACRPAAGGAVHTRGLDFACDIYIWAPLAGTCGVLLLSLVITLYC
KRGRKKLLYIFKQPFMRPVQTTQEEDGCSCRFPEEEEGGCELRVKFSRSA
DAPAYQQGQNQLYNELNLGRREEYDVLDKRRGRDPEMGGKPQRRKNPQEG
LYNELQKDKMAEAYSEIGMKGERRRGKGHDGLYQGLSTATKDTYDALHMQ
ALPPRGS GATNFSLLKQAGDVEENPGP MEFGLSWVFLVALLRGVQCYFGK
LESKLSVIRNLNDQVLFIDQGNRPLFEDMTDSDCRDNAPRTIFIISMYKD
SQPRGMAVTISVKCEKISTLSCENKIISFKEMNPPDNIKDTKSDIIFFQR
SVPGHDNKMQFESSSYEGYFLACEKERDLFKLILKKEDELGDRSIMFTVQ
NED

SEQ ID NO:14h5RM19C7-P2A-h2RF22C7-P2A-IL12B-IL12A (Figure 2B)MKHLWFFLLLVAAPRWVLSQVQLQESGPGLVKPSQTLSV
TCTVSGVSLPDYGVSWIRQPPRKGLEWLGVIWGSETTYYNSALKSRLTIS
KDNSKSQVSLKMSSVTAADTAVYYCAKHYYYGGSYAMDYWGQGTLVTVSS
GGGGSGGGGSGGGGSDIQQMTAQSPSSLSASVGDRVTITCRASQDISKYL N WYQQKPDGTVKLLIYHTSRLHSGVPSRFSGSGSGTDYSLTISSLQPEDI
ATYFCQQGNTLPYT FGGGTKVEIKGAP TTTPAPRPPTPAPTIASQPLSLR
PEACRPAAGGAVHTRGLDFACDIYIWAPLAGTCGVLLLSLVITLYCKRGR
KKLLYIFKQPFMRPVQTTQEEDGCSCRFPEEEEGGCELRVKFSRSADAPA
YQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPP
RAS GSGATNFSLLKQAGDVEENPGP MKHLWFFLLLVAAPRWVLSEVQLVE
SGGGLVQPGGSLRLSCAASGFTFSIYDMSWVRQAPGKGLEWVSYISSGGG
TTYYPDTVKGRFTISRDNAKNSLYLQMNSLRAEDTAVYYCARHSGYGSSY
GVLFAYWGQGTLVTGGGGSGGGGSGGGGSDIQMTQSPSSLSASVGDRVTI
TCRASQDISNYLNWYQQKPGKAPKLLIYYTSILHSGVPSRFSGSGSGTDF
TLTISSLQQEDFATYYCQQGNTLPWTFGGGTKVEIKPAGTTTPAPRPPTP
APTIASQPLSLRPEACRPAAGGAVHTRGLDFACDIYIWAPLAGTCGVLLL
SLVITLYCKRGRKKLLYIFKQPFMRPVQTTQEEDGCSCRFPEEEEGGCEL
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPRGSGATNFSLLKQAGDVEENPGP MEFGLSWVFLVALLR
GVQCIWELKKDVYVVELDWYPDAPGEMVVLTCDTPEEDGITWTLDQSSEV
LGSGKTLTIQVKEFGDAGQYTCHKGGEVLSHSLLLLHKKEDGIWSTDILK
DQKEPKNKTFLRCEAKNYSGRFTCWWLTTISTDLTFSVKSSRGSSDPQGV
TCGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHSY
FSLTFCVQVQGKSKREKKDRVFTDKTSATVICRKNASISVRAQDRYYSSS
WSEWASVPCSGGGGSGGGGSRNLPVATPDPGMFPCLHHSQNLLRAVSNML
QKARQTLEFYPCTSEEIDHEDITKDKTSTVEACLPLELTKNESCLNSRET
SFITNGSCLASRKTSFMMALCLSSIYEDLKMYQVEFKTMNAKLLMDPKRQ
IFLDQNMLAVIDELMQALNFNSETVPQKSSLEEPDFYKTKIKLCILLHAF
RIRAVTIDRVMSYLNAS

SEQ ID NO:15h5RM19scFv-linker-h2RF22C7-P2A-IL18 (F igure 2C)MKHLWFFLLLVAAPRWVLSQVQLQESGPGLVKPSQTLSVTC
TVSGVSLPDYGVSWIRQPPRKGLEWLGVIWGSETTYYNSALKSRLTISKD
NSKSQVSLKMSSVTAADTAVYYCAKHYYYGGSYAMDYWGQGTLVTVSSGG
GGSGGGGSGGGGSDIQQMTAQSPSSLSASVGDRVTITCRASQDISKYLNW
YQQKPDGTVKLLIYHTSRLHSGVPSRFSGSGSGTDYSLTISSLQPEDIAT
YFCQQGNTLPYTFGGGTKVElKPAGGGGSEVQLVESGGGLVQPGGSLRLS
CAASGFTFSIYDMSWVRQAPGKGLEWVSYISSGGGTTYYPDTVKGRFTIS
RDNAKNSLYLQMNSLRAEDTAVYYCARHSGYGSSYGVLFAYWGQGTLVTG
GGGSGGGGSGGGGSDIQMTQSPSSLSASVGDRVTITCRASQDISNYLNWY
QQKPGKAPKLLIYYTSILHSGVPSRFSGSGSGTDFTLTISSLQQEDFATY
YCQQGNTLPWTFGGGTKVEIKGAPTTTPAPRPPTPAPTIASQPLSLRPEA
CRPAAGGAVHTRGLDFACDIYIWAPLAGTCGVLLLSLVITLYCKRGRKKL
LYIFKQPFMRPVQTTQEEDGCSCRFPEEEEGGCELRVKFSRSADAPAYQQ
GQNQLYNELNLGRREEYDVLDKRRGRDPEMGGKPQRRKNPQEGLYNELQK
DKMAEAYSEIGMKGERRRGKGHDGLYQGLSTATKDTYDALHMQALPPRGS
GATNFSLLKQAGDVEENPGP MEFGLSWVFLVALLRGVQCYFGKLESKLSV
IRNLNDQVLFIDQGNRPLFEDMTDSDCRDNAPRTIFIISMYKDSQPRGMA
VTISVKCEKISTLSCENKIISFKEMNPPDNIKDTKSDIIFFQRSVPGHDN
KMQFESSSYEGYFLACEKERDLFKLILKKEDELGDRSIMFTVQNED

7. Examples of additional inducible genes:

SEQ ID NO:16h2RF22U1- P2A- IL18 (Figure 1; inducib le genes; antiCD19xCD3 bispecific antibody and IL1 8)MEFGLSWVFLVALLRGVQCEVQLVESGGGLVQPGGSLRLSCAASGFTF
SlYDMSWVRQAPGKGLEWVSYISSGGGTTYYPDTVKGRFTISRDNAKNSL
YLQMNSLRAEDTAVYYCARHSGYGSSYGVLFAYWGQGTLVTGGGGSGGGG
SGGGGSDIQMTQSPSSLSASVGDRVTITCRASQDISNYLNWYQQKPGKAP
KLLIYYTSILHSGVPSRFSGSGSGTDFTLTISSLQQEDFATYYCQQGNTL
PWTFGGGTKVElKPAGGGGGSEVQLVESGGGLVQPGGSLRLSCAASGYSF
TGYTMNWVRQAPGKGLEWVALINPYKGVSTYNQKFKDRFTISVDKSKNTA
YLQMNSLRAEDTAVYYCARSGYYGDSDWYFDVWGQGTLVTVSSGGGGSGG
GGSGGGGSDIQMTQSPSSLSASVGDRVTITCRASQDIRNYLNWYQQKPGK
APKLLIYYTSRLESGVPSRFSGSGSGTDYTLTISSLQPEDFATYYCQQGN
TLPWTFGQGTKVEIKPGS GATNFSLLKQAGDVEENPGP MEFGLSWVFLVA
LLRGVQCYFGKLESKLSVIRNLNDQVLFIDQGNRPLFEDMTDSDCRDNAP
RTIFIISMYKDSQPRGMAVTISVKCEKISTLSCENKIISFKEMNPPDNIK
DTKSDIIFFQRSVPGHDNKMQFESSSYEGYFLACEKERDLFKLILKKEDE
LGDRSIMFTVQNED

SEQ ID NO:17IL12B-linker-IL12A (Figure 1; inducibl e gene; fused IL12B and IL12A chains)MKHLWFFLLLVAA
PRWVLSIWELKKDVYVVELDWYPDAPGEMVVLTCDTPEEDGITWTLDQSS
EVLGSGKTLTIQVKEFGDAGQYTCHKGGEVLSHSLLLLHKKEDGIWSTDI
LKDQKEPKNKTFLRCEAKNYSGRFTCWWLTTISTDLTFSVKSSRGSSDPQ
GVTCGAATLSAERVRGDNKEYEYSVECQEDSACPAAEESLPIEVMVDAVH
KLKYENYTSSFFIRDIIKPDPPKNLQLKPLKNSRQVEVSWEYPDTWSTPH
SYFSLTFCVQVQGKSKREKKDRVFTDKTSATVICRKNASISVRAQDRYYS
SSWSEWASVPCSGGGGSGGGGSRNLPVATPDPGMFPCLHHSQNLLRAVSN
MLQKARQTLEFYPCTSEEIDHEDITKDKTSTVEACLPLELTKNESCLNSR
ETSFITNGSCLASRKTSFMMALCLSSIYEDLKMYQVEFKTMNAKLLMDPK
RQIFLDQNMLAVIDELMQALNFNSETVPQKSSLEEPDFYKTKIKLCILLH
AFRIRAVTIDRVMSYLNAS

What is claimed is:

1. A bispecific chimeric antigen receptor, comprising an extracellular domain linked to an intercellular domain through a linker, wherein the extracellular domain comprises a first scFv linked to a second scFv, wherein the first scFv domain and the second scFv domain each independently has affinity towards CD19 or CD22, wherein the first and the second scFv domain has affinity towards different antigens, and wherein the intercellular domain comprises a co-stimulatory endo-domain domain an a CD3ξ domain.

2. A dual specific chimeric antigen receptor complex, comprising
a first protein, comprising a first extracellular domain linked to a first intercellular domain through a first linker, wherein the first extracellular domain comprises a first scFv having affinity towards CD19 or CD22, and wherein the first intercellular domain comprises a JAK1 binding domain, and
a second protein, comprising a second extracellular domain linked to a second intercellular domain through a second linker, wherein the second extracellular domain comprises a second scFv having affinity towards CD 19 or CD22, and wherein the second intercellular domain comprises a JAK3 binding domain;
wherein the first scFv domain and the second scFv domain has affinity toward different tumor antigens.

3. The chimeric antigen receptor complex of claim 2, wherein the first intracellular domain comprises IL7Rα(CD127).

4. The chimeric antigen receptor complex of claim 2, wherein the first intracellular domain comprises intracellular domain of IL15Rβ(CD122), IL21Rα (CD360), or a combination thereof.

5. The chimeric antigen receptor complex of claim 2, wherein the first intracellular further comprises a first cytotoxic signaling domain linked to a JAK1 binding domain.

6. The chimeric antigen receptor complex of claim 5, wherein the first cytotoxic signaling domain comprises CD28, CD3ζ, CD137, OX40, CD27, ICOS, or a combination thereof.

7. The chimeric antigen receptor complex of claim 2, wherein the first scFv domain has an affinity toward CD19.

8. The chimeric antigen receptor complex of claim 2, wherein the second scFv domain has an affinity toward CD22.

9. The chimeric antigen receptor complex of claim 2, wherein the second intracellular domain comprises y(CD132).

10. The chimeric antigen receptor complex of claim 2, wherein the second intracellular domain further comprises a second cytotoxic signaling domain linked to a JAK3 binding domain.

11. The chimeric antigen receptor complex of claim 2, wherein the second cytotoxic domain comprises CD28, CD3ζ, CD137, OX40, CD27, ICOS, or a combination thereof.

12. The chimeric antigen receptor complex of claim 2, wherein the second intracellular domain comprises in tandem y(CD132), JAK3 binding domain, CD28, and CD3ζ.

13. The chimeric antigen receptor complex of claim 2, wherein the first intracellular domain is configured to dimerize with the second intracellular domain.

14. The chimeric antigen receptor complex of claim 2, wherein the first and the second linker comprises independently CD8.

15. The chimeric antigen receptor complex of claim 2, wherein the first and the second linker comprises independently a stalk and a transmembrane domain.

16. The chimeric antigen receptor complex of claim 13, wherein the stalk comprises CD8, Fc hinge, Fc CH2-CH3, TCRα, TCRβ, truncated IL7Ra (CD127), truncated IL15Rβ (CD122), IL15Rα (CD215), truncatedy (CD132), truncated IL21Rα (CD360), or a combination thereof.

17. The chimeric antigen receptor complex of claim 13, wherein the transmembrane domain comprises CD8, CD28, CD3ζ, CD3ε, CD3δ, CD3γ, CD3ζ, TCRα, TCRβ, IL15Rβ (CD122), γ(CD132), IL7Rα (CD127), IL21Rα (CD360), IL15Rα (CD215), or a combination of.

18. An open reading frame (ORF), comprising sequentially a nucleic acid encoding the first protein of claim 2, a nucleic acid encoding a ribosomal skipping sequence, and a nucleic acid encoding a second protein of claim 2.

19. An open reading frame (ORF), comprising sequentially CD22 scFv, a linker, CD22 scFv, and a chimeric antigen receptor domain.

20. A biomolecule complex, comprising the bispecific or dual chimeric antigen receptor of claim 1 bound to a CD19 antigen or a CD22 antigen.

21. The biomolecule complex of claim 22, wherein the first intracellular domain is dimerized with the second intracellular domain.

22. The biomolecule complex of claim 23, wherein JAK1 is dimerized with JAK3.

23. A non-viral vector, comprising, from 5′ to 3′, flanked by two transposons, a promoter, a first coding region comprising a gene for expressing a first artificial immunosurveillance chimeric antigen receptor (Al-CAR), a fourth coding region comprising a gene expressing a truncated CD20 or truncated EGFR safety target, followed by a polyA signal sequence.

24. The non-viral vector of claim 25, wherein the first promoter comprises a STAT, NFAT, or NF-kB inducible promoter.

25. The non-viral vector of claim 25, wherein the first coding region and the fourth coding region is linked by an IRES.

26. The non-viral vector of claim 25, wherein the first AI-CAR comprises CD19 CAR or CD22 CAR.

27. The non-viral vector of claim 25, further comprising a second coding region comprising a gene for expressing a second AI-CAR, intermediating the first coding region and the fourth coding region.

28. The non-viral vector of claim 29, wherein the first CAR comprises CD19 CAR and wherein the second region comprises CD22 CAR or CD20 CAR.

29. The non-viral vector of claim 30, further comprising a third coding region comprising a gene for expressing a third AI-CAR, intermediating the second coding region and the fourth coding region.

30. The non-viral vector of claim 31, wherein the first CAR comprises CD19scFv, wherein the second CAR comprising CD22 scFv or CD20 scFv, and wherein the first CAR is linked to the second CAR through a linker.


SEQUENCE LISTING (17 sequences)

SEQ ID NO: 1 - protein, 488 aa, Artificial Sequence
  Synthesized
MKHLWFFLLLVAAPRWVLSQVQLQESGPGLVKPSETLSLTCTVSGGSIPDYGVSWIRQPPGKGLEWIGVIWGSETTYYNSALKSRVTISVDTSKNQFSLKLSSVTAADTAVYYCARHYYYGGSYAMDYWGQGTSLTVSSGGGGSGGGGSGGGSGDIQMTQSPSSLSASVGDRVTITCRASQDISKYLNWYQQKPGKAPKLLIYHTSRLHSGVPSRFSGSGSGTDFTLTISSLQPEDFATYYCQQGNTLPYTFGGGTKVEITGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - protein, 488 aa, Artificial Sequence
  Synthesized
MKHLWFFLLLVAAPRWVLSDIQMTQSPSSLSASVGDRVTITCRASQDISKYLNWYQQKPGGAPKLLIYHTSRLHSGVPSRFSGSGSGTDFTFTISSLQQEDIATYYCQQGNTLPYTFGGGTKVEIKGGGGSGGGGSGGGGSQVQLQESGPGLVKPSQTLSLTCTVSGVSLPDYGVSWIRQHPGKGLEWIGVIWGSETTYYNSALKSRVTISVDTSKNQFSLKLSSVTAADTAVYYCARHYYYGGSYAMDYWGQGTLVTVSSGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 3 - protein, 488 aa, Artificial Sequence
  Synthesized
MKHLWFFLLLVAAPRWVLSQVQLQESGPGLVKPSQTLSLTCTVSGVSIPDYGVSWIRQHPGKGLEWIGVIWGSETTYYNSALKSRVTISVDTSKNQFSLKLSSVTAADTAVYYCARHYYYGGSYAMDYWGQGTLVTVSSGGGGSGGGGSGGGGSDIQMTQSPSSLSASVGDRVTITCRASQDISKYLNWYQQKPGKAPKLLIYHTSRLHSGVPSRFSGSGSGTDFTFTISSLQQEDIATYYCQQGNTLPYTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 4 - protein, 490 aa, Artificial Sequence
  Synthesized
MKHLWFFLLLVAAPRWVLSEVQLVESGGGLVQPGGSLRLSCAASGFAFSIYDMSWVRQAPGKGLEWVSYISSGGGTTYYPDTVKGRFTISRDNAKNSLYLQMNSLRAEDTAVYYCARHSGYGSSYGVLFAYWGQGTLVTVSGGGGSGGGGSGGGGSDIQMTQSPSSLSASVGDRVTITCRASQDISNYLNWYQQKPGKAPKLLIYYTSILHSGVPSRFSGSGSGTDFTFTISSLQQEDIATYYCQQGNTLPWTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - protein, 488 aa, Artificial Sequence
  Synthesized
MKHLWFFLLLVAAPRWVLSEVQLVESGGGLVQPGGSLRLSCAASGFTFSIYDMSWVRQAPGKGLEWVSYISSGGGTTYYPDTVKGRFTISRDNAKNSLYLQMNSLRAEDTAVYYCARHSGYGSSYGVLFAYWGQGTLVTGGGGSGGGGSGGGGSDIQMTQSPSSLSASVGDRVTITCRASQDISNYLNWYQQKPGKAPKLLIYYTSILHSGVPSRFSGSGSGTDFTLTISSLQQEDFATYYCQQGNTLPWTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 6 - protein, 751 aa, Artificial Sequence
  Synthesized
MKHLWFFLLLVAAPRWVLSQVQLQESGPGLVKPSQTLSLTCTVSGVSLPDYGVSWIRQHPGKGLEWIGVIWGSETTYYNSALKSRVTISVDTSKNQFSLKLSSVTAADTAVYYCARHYYYGGSYAMDYWGQGTLVTVSSGGGGSGGGGSGGGGSDIQMTQSPSSLSASVGDRVTITCRASQDISKYLNWYQQKPGGAPKLLIYHTSRLHSGVPSRFSGSGSGTDFTFTISSLQQEDIATYYCQQGNTLPYTFGGGTKVEIKPAGGGEPKSSDKTHTGGASEVQLVESGGGLVQPGGSLRLSCAASGFAFSIYDMSWVRQAPGKGLEWVSYISSGGGTTYYPDTVKGRFTISRDNAKNSLYLQMNSLRAEDTAVYYCARHSGYGSSYGVLFAYWGQGTLVTVSGGGGSGGGGSGGGGSDIQMTQSPSSLSASVGDRVTITCRASQDISNYLNWYQQKPGKAPKLLIYYTSILHSGVPSRFSGSGSGTDFTFTISSLQQEDIATYYCQQGNTLPWTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 7 - protein, 749 aa, Artificial Sequence
  Synthesized
MKHLWFFLLLVAAPRWVLSQVQLQESGPGLVKPSQTLSLTCTVSGVSIPDYGVSWIRQHPGKGLEWIGVIWGSETTYYNSALKSRVTISVDTSKNQFSLKLSSVTAADTAVYYCARHYYYGGSYAMDYWGQGTLVTVSSGGGGSGGGGSGGGGSDIQMTQSPSSLSASVGDRVTITCRASQDISKYLNWYQQKPGKAPKLLIYHTSRLHSGVPSRFSGSGSGTDFTFTISSLQQEDIATYYCQQGNTLPYTFGGGTKVEIKPAGGGEPKSSDKTHTGGASEVQLVESGGGLVQPGGSLRLSCAASGFTFSIYDMSWVRQAPGKGLEWVSYISSGGGTTYYPDTVKGRFTISRDNAKNSLYLQMNSLRAEDTAVYYCARHSGYGSSYGVLFAYWGQGTLVTGGGGSGGGGSGGGGSDIQMTQSPSSLSASVGDRVTITCRASQDISNYLNWYQQKPGKAPKLLIYYTSILHSGVPSRFSGSGSGTDFTLTISSLQQEDFATYYCQQGNTLPWTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - protein, 979 aa, Artificial Sequence
  Synthesized
MKHLWFFLLLVAAPRWVLSQVQLQESGPGLVKPSQTLSLTCTVSGVSLPDYGVSWIRQHPGKGLEWIGVIWGSETTYYNSALKSRVTISVDTSKNQFSLKLSSVTAADTAVYYCARHYYYGGSYAMDYWGQGTLVTVSSGGGGSGGGGSGGGGSDIQMTQSPSSLSASVGDRVTITCRASQDISKYLNWYQQKPGGAPKLLIYHTSRLHSGVPSRFSGSGSGTDFTFTISSLQQEDIATYYCQQGNTLPYTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPEVQLVESGGGLVQPGGSLRLSCAASGFTFSIYDMSWVRQAPGKGLEWVSYISSGGGTTYYPDTVKGRFTISRDNAKNSLYLQMNSLRAEDTAVYYCARHSGYGSSYGVLFAYWGQGTLVTGGGGSGGGGSGGGGSDIQMTQSPSSLSASVGDRVTITCRASQDISNYLNWYQQKPGKAPKLLIYYTSILHSGVPSRFSGSGSGTDFTLTISSLQQEDFATYYCQQGNTLPWTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - protein, 326 aa, Artificial Sequence
  Synthesized
MKHLWFFLLLVAAPRWVLSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGSGATNFSLLKQAGDVEENPGPQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 10 - protein, 326 aa, Artificial Sequence
  Synthesized
MKHLWFFLLLVAAPRWVLSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGSGATNFSLLKQAGDVEENPGPGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 11 - protein, 1026 aa, Artificial Sequence
  Synthesized
MKHLWFFLLLVAAPRWVLSQVQLQESGPGLVKPSQTLSVTCTVSGVSLPDYGVSWIRQPPRKGLEWLGVIWGSETTYYNSALKSRLTISKDNSKSQVSLKMSSVTAADTAVYYCAKHYYYGGSYAMDYWGQGTLVTVSSGGGGSGGGGSGGGGSDIQMTQSPSSLSASVGDRVTITCRASQDISKYLNWYQQKPDGTVKLLIYHTSRLHSGVPSRFSGSGSGTDYSLTISSLQPEDIATYFCQQGNTLPYTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRASGSGATNFSLLKQAGDVEENPGPMEFGLSWVFLVALLRGVQCEVQLVESGGGLVQPGGSLRLSCAASGFTFSIYDMSWVRQAPGKGLEWVSYISSGGGTTYYPDTVKGRFTISRDNAKNSLYLQMNSLRAEDTAVYYCARHSGYGSSYGVLFAYWGQGTLVTGGGGSGGGGSGGGGSDIQMTQSPSSLSASVGDRVTITCRASQDISNYLNWYQQKPGKAPKLLIYYTSILHSGVPSRFSGSGSGTDFTLTISSLQQEDFATYYCQQGNTLPWTFGGGTKVEIKPAGGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKP

SEQ ID NO: 12 - protein, 688 aa, Artificial Sequence
  Synthesized
MKHLWFFLLLVAAPRWVLSQVQLQESGPGLVKPSQTLSVTCTVSGVSLPDYGVSWIRQPPRKGLEWLGVIWGSETTYYNSALKSRLTISKDNSKSQVSLKMSSVTAADTAVYYCAKHYYYGGSYAMDYWGQGTLVTVSSGGGGSGGGGSGGGGSDIQMTQSPSSLSASVGDRVTITCRASQDISKYLNWYQQKPDGTVKLLIYHTSRLHSGVPSRFSGSGSGTDYSLTISSLQPEDIATYFCQQGNTLPYTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRASGSGATNFSLLKQAGDVEENPGPMEFGLSWVFLVALLRGVQCYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 13 - protein, 1198 aa, Artificial Sequence
  Synthesized
MKHLWFFLLLVAAPRWVLSQVQLQESGPGLVKPSQTLSVTCTVSGVSLPDYGVSWIRQPPRKGLEWLGVIWGSETTYYNSALKSRLTISKDNSKSQVSLKMSSVTAADTAVYYCAKHYYYGGSYAMDYWGQGTLVTVSSGGGGSGGGGSGGGGSDIQMTQSPSSLSASVGDRVTITCRASQDISKYLNWYQQKPDGTVKLLIYHTSRLHSGVPSRFSGSGSGTDYSLTISSLQPEDIATYFCQQGNTLPYTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRASGSGATNFSLLKQAGDVEENPGPMKHLWFFLLLVAAPRWVLSEVQLVESGGGLVQPGGSLRLSCAASGFTFSIYDMSWVRQAPGKGLEWVSYISSGGGTTYYPDTVKGRFTISRDNAKNSLYLQMNSLRAEDTAVYYCARHSGYGSSYGVLFAYWGQGTLVTGGGGSGGGGSGGGGSDIQMTQSPSSLSASVGDRVTITCRASQDISNYLNWYQQKPGKAPKLLIYYTSILHSGVPSRFSGSGSGTDFTLTISSLQQEDFATYYCQQGNTLPWTFGGGTKVEIKPAGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMEFGLSWVFLVALLRGVQCYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 14 - protein, 1554 aa, Artificial Sequence
  Synthesized
MKHLWFFLLLVAAPRWVLSQVQLQESGPGLVKPSQTLSVTCTVSGVSLPDYGVSWIRQPPRKGLEWLGVIWGSETTYYNSALKSRLTISKDNSKSQVSLKMSSVTAADTAVYYCAKHYYYGGSYAMDYWGQGTLVTVSSGGGGSGGGGSGGGGSDIQMTQSPSSLSASVGDRVTITCRASQDISKYLNWYQQKPDGTVKLLIYHTSRLHSGVPSRFSGSGSGTDYSLTISSLQPEDIATYFCQQGNTLPYTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRASGSGATNFSLLKQAGDVEENPGPMKHLWFFLLLVAAPRWVLSEVQLVESGGGLVQPGGSLRLSCAASGFTFSIYDMSWVRQAPGKGLEWVSYISSGGGTTYYPDTVKGRFTISRDNAKNSLYLQMNSLRAEDTAVYYCARHSGYGSSYGVLFAYWGQGTLVTGGGGSGGGGSGGGGSDIQMTQSPSSLSASVGDRVTITCRASQDISNYLNWYQQKPGKAPKLLIYYTSILHSGVPSRFSGSGSGTDFTLTISSLQQEDFATYYCQQGNTLPWTFGGGTKVEIKPAGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMEFGLSWVFLVALLRGVQCIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 15 - protein, 935 aa, Artificial Sequence
  Synthesized
MKHLWFFLLLVAAPRWVLSQVQLQESGPGLVKPSQTLSVTCTVSGVSLPDYGVSWIRQPPRKGLEWLGVIWGSETTYYNSALKSRLTISKDNSKSQVSLKMSSVTAADTAVYYCAKHYYYGGSYAMDYWGQGTLVTVSSGGGGSGGGGSGGGGSDIQMTQSPSSLSASVGDRVTITCRASQDISKYLNWYQQKPDGTVKLLIYHTSRLHSGVPSRFSGSGSGTDYSLTISSLQPEDIATYFCQQGNTLPYTFGGGTKVEIKPAGGGGSEVQLVESGGGLVQPGGSLRLSCAASGFTFSIYDMSWVRQAPGKGLEWVSYISSGGGTTYYPDTVKGRFTISRDNAKNSLYLQMNSLRAEDTAVYYCARHSGYGSSYGVLFAYWGQGTLVTGGGGSGGGGSGGGGSDIQMTQSPSSLSASVGDRVTITCRASQDISNYLNWYQQKPGKAPKLLIYYTSILHSGVPSRFSGSGSGTDFTLTISSLQQEDFATYYCQQGNTLPWTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMEFGLSWVFLVALLRGVQCYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 16 - protein, 712 aa, Artificial Sequence
  Synthesized
MEFGLSWVFLVALLRGVQCEVQLVESGGGLVQPGGSLRLSCAASGFTFSIYDMSWVRQAPGKGLEWVSYISSGGGTTYYPDTVKGRFTISRDNAKNSLYLQMNSLRAEDTAVYYCARHSGYGSSYGVLFAYWGQGTLVTGGGGSGGGGSGGGGSDIQMTQSPSSLSASVGDRVTITCRASQDISNYLNWYQQKPGKAPKLLIYYTSILHSGVPSRFSGSGSGTDFTLTISSLQQEDFATYYCQQGNTLPWTFGGGTKVEIKPAGGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKPGSGATNFSLLKQAGDVEENPGPMEFGLSWVFLVALLRGVQCYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 17 - protein, 532 aa, Artificial Sequence
  Synthesized
MKHLWFFLLLVAAPRWVLSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS